(19) United States
(12) Patent Application Publication
(10) Pub. No.: US 2011/0020862 A1
(43) Pub. Date: Jan. 27, 2011

(54) Biological solids processing system and method

(75) Inventors: Yves Audebert, Los Gatos, CA (US); Thomas Jahn, Redwood City, CA (US); Ronald Mosso, Fremont, CA (US); Michael Oda, San Francisco, CA (US)

Correspondence Address: Herbert Burkard, SUITE 211, 405 EL CAMINO REAL, MENLO PARK, CA, 94025, US

(73) Assignee: Kainos Power LLC.

(21) Appl. No.: 12/802,011

(22) Filed: May 27, 2010

Related U.S. Application Data
(60) Provisional application No. 61/217,322, filed on May 29, 2009.

(51) Int. Cl. C12P 1/00; C12M 1/00

(52) U.S. Cl. 435/41; 435/290.1

(57) ABSTRACT

The consumption of organic solids with anaerobic digestion to generate usable gases including methane is made more efficient by maintaining the ideal digestion temperature, which is attained by combining the anaerobic digestion process with a halogen digester which produces heat energy and hydrogen gas. With a given biological feedstock four outputs can be generated (methane, hydrogen, electricity, and heat) in the ratio that makes the most economical sense. The process also provides a significant reduction in volume of output solids. The halogen oxidation process can be used on all the anaerobic digester effluent to extract more energy and oxidize a wet feedstock. If there are solids which are not easily digested with the anaerobic process, these solids can be diverted to the halogen digester to derive more energy from the feedstock. Pathogens common to other anaerobic digester effluents are removed. The mixture of methane and hydrogen gas can be compressed to produce an enriched compressed natural gas (CNG) with a variety of uses.

### CROSS-REFERENCE TO RELATED APPLICATIONS

This application claims priority from provisional application 61/217,322 filed May 29, 2009

### STATEMENT REGARDING FEDERALLY SPONSORED RESEARCH (IF APPLICABLE)

None.

### BACKGROUND OF THE INVENTION

Anaerobic digestion of biological waste to produce biofuels is a growing area of interest as concerns about greenhouse gas emissions grow and the use of, and demand for, alternative and renewable energy sources increases. This form of digestion is the process in which an environment free of oxygen allows certain microorganisms to flourish, consuming biological solids and creating biogas that contains a considerable amount of methane. If not collected, the bio-gas enters the atmosphere as a greenhouse gas with a much stronger greenhouse effect than carbon dioxide. If captured, the biogas can be used to generate heat and/or electricity or the system can be used to eliminate solid wastes. There are a number of limitations in the current implementations of the anaerobic digestion technology. In particular, anaerobic digestion is sensitive to temperature, and if the temperature of the biological solids is too low, then the digestion process will either slow or even halt completely. Slower digestion times require longer retention times that lead to higher costs due to increased digestion tank size. Many anaerobic digestion systems utilize much of the energy produced by the system in the form of heat just to maintain digester temperature and digester function. These temperature requirements normally limit the areas where anaerobic digestion is feasible to a warmer, more temperature stable environment. With a given feedstock there are also some solids that are not easily digested with the anaerobic process. These solids can be diverted to the chemical oxidation digester segment of the present invention that enables the anaerobic digester to run more efficiently as well as provide the potential to derive more energy from the feedstock. The process of the present invention allows for warm and stable digestion temperatures, enabling the process to be carried out in colder climates; produces an effluent with fewer or no pathogens; has shorter retention times for feed stock; and provides a method to extract an increased amount of energy per unit of feedstock.

### SUMMARY OF THE INVENTION

The present invention combines biological digestion with chemical oxidation to optimize the extraction of energy from biological waste. The terms “biowaste”, “biological waste”, or “biological feedstock” as used herein contemplates any organic solid that can be digested by aerobic or anaerobic bacteria, including plant, animal and/or mixed municipal waste. The consumption of biowaste, such as manure and food processing waste, with anaerobic digestion to generate combustible and other usable gases, including hydrogen and methane, can be made more efficient by maintaining the optimum anaerobic digestion temperature. Although a preferred embodiment of the present invention utilizes anaerobic digestion and it will be described in this context, it is also applicable to aerobic digestion or a combination in sequence of aerobic and anaerobic digestion. We have found that the desired higher digestion temperatures can be attained by combining the anaerobic digestion process with a subsequent halogen digester oxidation process which produces both usable heat energy and hydrogen from partially digested or even undigested organic solids. Numerous psychrophilic, mesophilic and thermophilic bacteria families of the Archaea genera are well known to be useful in anaerobic digestion. We have found that one of the most useful fermentative bacteria in the processing of thermophilic waste sludge is the Thermoanaerobacterium genus. An example is the species aotearoense of the Clostridia class. For aerobic digestion major bacterial groups in the beginning of the composting process are mesophilic organic acid producing bacteria such as Lactobacillus spp. and Acetobacter spp. Later, at the thermophilic stage, Gram-positive bacteria such as Bacillus spp. and Actinobacteria, become dominant. Feedstock commonly contains biological solids that are both digested through anaerobic and aerobic digestion as well as solids that require extensive treatment and retention times or even cannot be successfully processed. These low volatility solids add unnecessary material to the digester that slows the overall conversion of the feedstock to energy and create a buildup of materials. By not including separation the size of the digester needs to be larger to retain these solids and must also accommodate longer retention times. Maintenance costs of the facility increase and downtime is required when the materials need to be removed. An example of solids that causes problems to anaerobic digester plants is lignocellulose. This material would be sent directly to the chemical process where they are easily digested.

Anaerobic digestion is a biological process that works optimally at stable temperatures for given organisms. Heat loss can cause the digestion process to slow and result in longer retention times that increases the physical size of the digester needed. Unconditioned feedstock can cause a shock to the bacterial colonies and slow or spoil the digestion. Many digester models are oversized so that the thermal shock of adding fresh feedstock is lessened. If the feedstock is properly conditioned by separation of less easily digested solids out of the incoming stream as well as normalizing the temperature to the same thermal conditions of the digestion tank contents then this will result in a more predictable and efficient digestion process. The retention times will decrease and the overall size and capital costs will decrease as well. The capabilities of the system and process of the present invention are such that with a given biological feedstock, the combined system can generate multiple alternative useful outputs (i.e., methane, hydrogen, electricity, and heat) with the flexibility for the particular plant owner/operator to focus on which output is most desired. The advantages of the system and process of the present invention are not limited to the energy output of the system. The process provides a significant reduction in the volume of the waste solids thereby reducing costs associated with disposal or distribution. The halogen oxidation process can be applied to the digestate from the aerobic and/or anaerobic digester to extract more energy and effectively oxidize even a wet digestate feedstock. Pathogens common to existing anaerobic digester effluents are destroyed by both the heat and oxidation resulting from the halogen oxidation process. The residual solids output from the halogen digester comprise a substantially or totally pathogen free, micronutrient-rich ash that is useful as a fertilizer. The liquid portion, which contains the aqueous hydrogen halide acid, is advantageously passed on to an electrolysis chamber for generating hydrogen and regenerating the halogen for recycling and reuse in the oxidation process.

A useful application of the combined output of the anaerobic bio-digester and the halogen digester is the production of an enriched natural gas for use in the transportation industry. The methane and hydrogen gas mixture produced by the combined process of the present invention produces significantly reduced harmful emissions over conventional fuels such as diesel and gasoline. The mixture of methane and hydrogen can be readily compressed to produce an enriched compressed natural gas (CNG) having 95% less emissions per Gallon Gasoline Equivalent (GGE) than diesel fuel or alternatively can be sold to a gas pipeline utility.

Alternatively, the gases created by the system can be used in conjunction with an electricity generator such as a fuel cell or internal combustion engine, combustion turbine or the like connected to an electrical generator to produce electricity that can be fed to the grid. The flexibility of the output of the process of the present invention allows the system to offset electricity and heating costs for both the digestion plant and/or also daily farm or other plant operations while simultaneously producing sellable products to further offset costs and/or generate profit.

The following steps describe alternative aspects and embodiments of the process and apparatus of the present invention:
I. The bio-waste feedstock material can optionally undergo a filtering process where solids that are difficult to digest are separated out to distribute the materials to the halogen digester that can most efficiently handles the solids.
II. The bio-waste feedstock material is preheated by heat generated by the chemical process, heat from the electricity generator or any combination of the preceding.
III. Alternatively, the bio-waste feedstock first enters into an aerobic digester and is aerated (and in very cold climates pre-heated) until the temperature of the feedstock reaches optimal temperatures for anaerobic digestion as a result of the exothermic aerobic reaction. (This temperature rise can be accelerated by exchanging the heat from the warm anaerobic digester liquid effluent or the heat generated by the chemical process, electricity generator or any combination of these heat sources).
IV. The feedstock is then transferred to the anaerobic digester, in which the elevated temperature increases the rate at which the suspended solids are anaerobically digested to yield a mixture of gases which can include, methane (CH₄), carbon dioxide (CO₂), H₂O, NH₃, H₂S, mercaptans and multi-carbon hydrocarbons, as well as partially digested feedstock effluent.
V. The gases are collected and then any NH₃, mercaptans and H₂S are removed with a scrubber.
VI. The effluent from the anaerobic digester (“digestate”) is preferably filtered if necessary to achieve the appropriate ratio of water to solids of approximately 40% for the halogen digester. The removed warm liquid can first be sent to a heat exchanger as indicated in step II.
VII. The digestate is then reacted (oxidized) with a halogen to produce aqueous hydrogen halide, a sterile micronutrient fertilizer, carbon dioxide and usable heat. In some cases additional fresh feedstock may also be input to the halogen reactor. The halogen can be either Chlorine, Bromine, or Iodine (Cl₂, Br₂, I₂). Bromine is preferred because of its significantly lower volatility relative to chlorine since it is a liquid at normal ambient temperature. Additionally, Bromine is soluble in aqueous HBr, which facilitates processing. Iodine is also soluble in aqueous HI. The halogen oxidation is an exothermic reaction.
VIII. The heated aqueous HBr solution from the halogen digester is then passed through an electrolysis chamber where the HBr is separated into H₂ and Br₂. The elevated temperature allows the use of less electricity to produce the hydrogen gas and regenerate the halogen for reuse. 2HBr→H₂+Br₂
IX. Any excess heat from the electrolysis chamber is transferred back to the anaerobic digester unit, for example by using a heat exchanger to regulate the temperature of the biological activity and maintain stable temperatures.
X. There are preferably scrubbers for the output of both the halogen digester and the electrolysis chamber to ensure minimal loss of reactants and also to remove any sulfur compounds and/or ammonia from the gaseous output.
XI. The solids from the halogen digester can be collected (for example by filtration) for use as a sterile micronutrient fertilizer.
XII. The hydrogen gas from Step VII and methane gas from Step IV can be combined to create a desirable combustible, clean burning gas mixture for compressed and/or natural gas pipeline applications.
XIII. If pure hydrogen is the desired output it can be readily recovered in pure form from the electrolysis process and utilized.
XIV. Alternatively, the hydrogen and/or methane gas mixture can also be fed to a fuel cell or coupled to an internal combustion engine or turbine coupled with an electricity generator to produce electricity and additional heat if desired.
XV. The hydrogen and methane gases can be directly fired to produce a large quantity of heat. In this case it may not be necessary to separate the gases from the anaerobic digestion as indicated in step IV.
XVI. Additional heat energy for warming the feedstock can be provided through use of a solar hot water heater.

### BRIEF DESCRIPTION OF THE DRAWINGS

The following numbers shown in the Figures indicate the following components or products.
1. Biological digestion system
2. Biowaste feedstock
3. Gases generated by biodigester 1 (CH₄, H₂S, CO₂, etc.)
4. Heat required by biodigester 1 to maintain ideal operating temperature (since anaerobic digestion is an endothermic process)
5. Electricity generator system
6. Electricity from generator 5. Some can be diverted for plant processing such as use in the electrolysis in process 9
7. Halogen—Electrolysis System
8. Hydrogen produced by the Halogen—Electrolysis process 7
9. Electricity required by the Halogen—Electrolysis process 7
10. Excess heat produced by process 7
11. Halogen reactor
12. Electrolysis unit, converting the halogen acid produced by reactor 11 into elemental halogens.
13. Electrolysis unit output, Br₂ I₂
14. Heat produced by exothermic reaction of halogen reaction
15. Heat exchange and distribution system, capturing heat from reactor output to bring HBr to optimal electrolysis temperature
16. Ashes produced by halogen reactor 11
17. Feedstock input to halogen process 7 (or system)
18. Electricity Generator (e.g. Fuel cell)
19. Scrubber to remove H₂S from gas flow 3 before feeding it to the electricity generator 18
20. Heat generated by the electricity generator 18
21. The additional heat required by the anaerobic digester to operate at optimal temperature.
22. Enriched compressed natural gas resulting from mixing the H₂ gas flow 8 with the methane gas flow
23. Scrubber to remove CO₂ and H₂S from gas flow 3
24. Methane gas flow out of scrubber 23
25. Mixing—Compression system to mix H₂ with methane with predefined ratio and compress the combination to produce CNG
26. Aerobic digester used to preprocess feedstock 2
27. Heat exchange and distribution system, recovering heat from anaerobic digester effluent (or excess effluent) and transferring heat to feedstock 2
28. Heat recovered from anaerobic digester effluent 29 (or excess effluent) and transferred to feedstock 2
29. Biodigestion effluent waste
30. Solar convector capturing heat from solar energy
31. Heat exchange and distribution system, recovering heat from solar convector and transferring heat to feedstock 2
32. Heat recovered from solar convector 30 and transferred to feedstock 2
33. Compression System for Hydrogen
34. Hydrogen Gas Output
35. Filter used to separate solids from the feedstock to different streams
36. Heat recovered from item 10 and 20 are used to preheat the incoming feedstock
37. Conditioned feedstock containing solids for anaerobic digestion
38. Filtered feedstock containing solids for halogen digestion
39. Biological digester solid and liquid effluent
40. Biological digester gaseous output
41. Mixture of hydrogen halide and water

All of FIGS. 1A through 6 are in block diagram form

FIG. 1A shows a prior art anaerobic biological digestion system.

FIG. 1B shows the use of the output of a prior art anaerobic biological digestion system of FIG. 1A to power an electric generator.

FIG. 2A shows a prior art halogen electrolysis system wherein the digestion of organic material by halogen is effected followed by electrolysis of the resulting hydrogen halide.

FIG. 2B shows the system of FIG. 2A in greater detail.

FIG. 3 shows the overall process of the present invention.

FIG. 4A shows a first embodiment of the present invention wherein the system produces hydrogen and methane which are used to fuel an electricity generator.

FIG. 4B shows a second embodiment of the present invention wherein the hydrogen and methane gas produced are scrubbed and combined for use as a fuel such as in a vehicle.

FIG. 4C shows a third embodiment of the present invention which is configured to maximize the production of hydrogen gas.

FIG. 5A shows an embodiment of the present invention which is designed to achieve maximum efficiency.

FIG. 5B shows an embodiment of the present invention which utilizes a solar heater to preheat the digester feedstock.

FIG. 6 shows in detail the electrolysis system utilized in the present invention.

### DETAILED DESCRIPTION OF THE INVENTION

The consumption of organic solids such as manure and food processing waste (2) by biological digestion system (1) that is anaerobic to generate usable gases (3) including methane can be accelerated by maintaining an anaerobic digestion temperature that is optimal for the particular combination of bacteria present in the digester. A vast quantity of biological waste is available but ambient temperatures at many locations where these wastes are generated are so low that additional heat is required for efficient operation of digesters to process those wastes. In fact, in many bio-digester facilities located in colder regions, the anaerobic digestion process will stop completely below a certain ambient temperature due to heat loss. If the expected anaerobic digester facility output is to be used for heat, electricity and/or compressed fuel obviating the interruption of this resource prevents adversely affecting operations. Keeping the temperatures optimal for anaerobic digestion can allow for a consistent output of gaseous product. In addition, the desired throughput can be achieved with a smaller digester unit if the optimum digestion temperature is maintained. This result lowers capital costs and allows a smaller plant footprint. We have found that higher temperatures can be readily attained by combining the anaerobic digestion process with a halogen digestion system (7), which produces heat energy during the exothermic oxidation of the organic solids. The use of a heat exchanger (15) can redirect the energy from this exothermic output (10) to preheat the influent organic materials (feedstock) (2) as well as heat the biological digester (1). The feedstock for the halogen digestion system (17) can be either fresh organic waste (2) or the effluent from the anaerobic digester (29) as feedstock for the halogen digester. Use of the anaerobic digester effluent (29) increases the total amount of energy accessed per unit of feedstock and at the same time sterilizes the output to prevent the proliferation of pathogens such as bacteria and viruses. The halogen digestion system (7) encompasses a reactor (11) and advantageously an electrolysis chamber (12). The hydrogen halide (13) produced from the oxidation of the feedstock in the reactor (11) can be passed through an electrolysis chamber (12) to produce hydrogen gas (8) and regenerate the halogen for recycling into the halogen digester. The electrolysis of the hydrogen halide to produce hydrogen gas and halogen requires significantly less electricity than the similar electrolysis process using water to produce hydrogen.

The feedstock contains a variety of solids including some which are hard to digest with anaerobic digestion such as lignocellulose as well as biologically inert materials. The incoming feedstock (2) can be sent through a filter (35) and the solids that are difficult or impossible to undergo anaerobic digestion (38) can be diverted directly to the chemical digester (5). This allows the solids best suited for the anaerobic digestion (37) to be preheated (36) so the conversion of solids to energy is more efficient. Another result is the drastically reduced retention times and lower maintenance costs for stirring and cleaning of built up materials in the anaerobic digester (1).

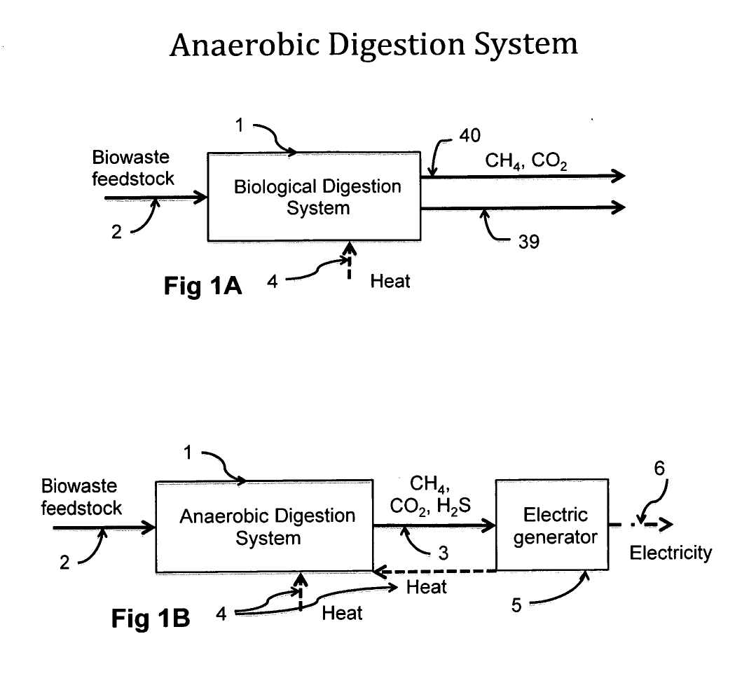

FIG. 1A shows a simple biological digestion process, wherein biowaste feedstock (2) is fed to a biological digestion system (1), which may use aerobic or anaerobic bacteria to break down the incoming organic waste. In the case where anaerobic bacteria are used, the mass leaving the digester is comprised of two streams, one stream is a solid-liquid slurry of digester effluent (39), while the other stream (40) is a gaseous stream containing mostly methane and carbon dioxide, as well as some other compounds. Heat (4) is typically required to maintain optimum conditions for anaerobic bacteria to digest the feedstock. This process is well known in the art and broadly practiced around the world.

FIG. 1B shows the addition of an electric generator (5) to the simple system (1) of FIG. 1A. In this system, the chemical energy in the methane (and other combustible components which may be present) in the gaseous effluent stream (3) from the digester, is recovered by conversion to heat (4) and electric energy (6) in an electric generator (5). The electric generator may be a fuel cell, which directly converts the chemical energy to electricity and heat, or it may be in internal combustion engine or combustion turbine that first converts the chemical energy to heat and mechanical energy, then to electrical energy in an electrical generator. In addition, heat from the imperfect conversion of chemical energy to electrical energy may be returned to the biodigestion process to maintain optimum conditions. However, additional heat beyond what is provided by the electric generator is required to maintain optimum conditions, particularly in colder climates. This combination of anaerobic digestion of biological waste with electric generation is known in the art and practiced commercially today.

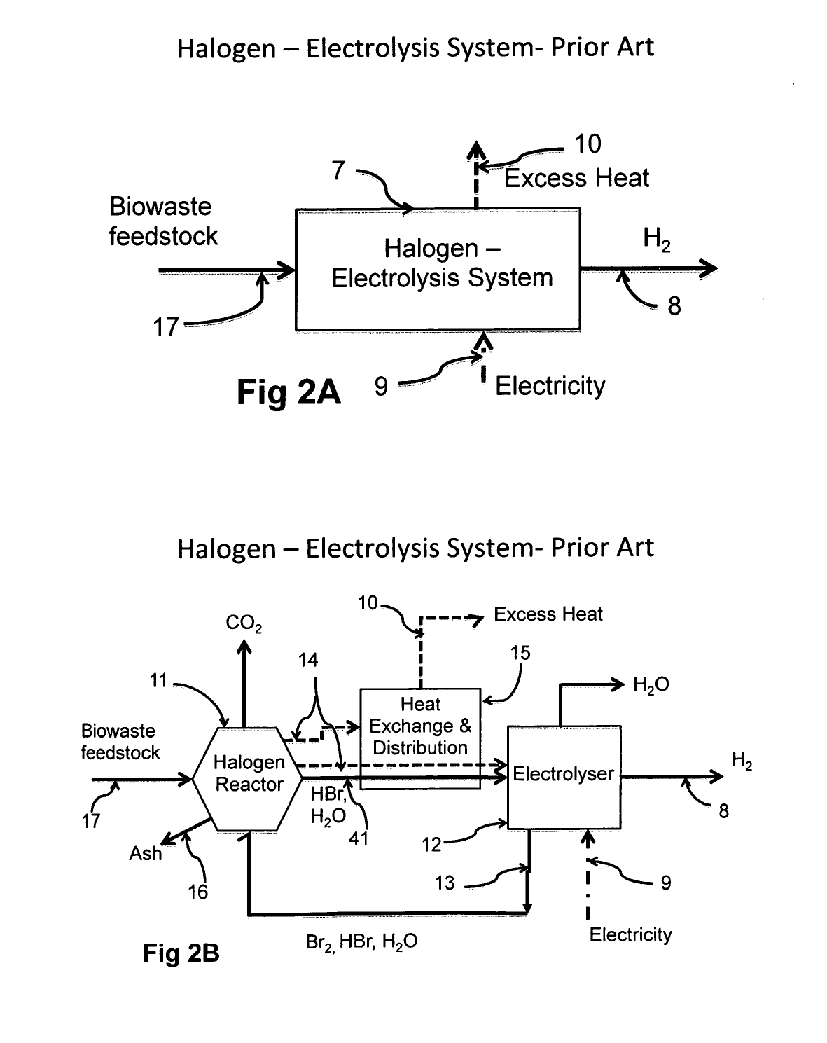

FIG. 2A is an overall representation of the halogen electrolysis system (7), wherein halogen digestion of organic material takes place in a halogen reactor. From an overall perspective, biowaste feed stock (17), which contains organic material, enters the halogen electrolysis system and is converted internally to a hydrogen halide and carbon dioxide. With the input of electrical energy (9), the hydrogen halide is electrolyzed into hydrogen gas and the dihalide. The dihalide is returned to the internal halogen reactor to digest more organic material and the hydrogen is removed at high purity as a valuable product (8). The halogen digestion of organic material is strongly exothermic and a significant amount of excess heat (10) is released from the process.

FIG. 2B shows the halogen electrolysis system in greater detail. Biowaste feedstock (17) enters the halogen reactor (11) where halogen digestion of the feedstock takes place. Carbon dioxide is released as a gas from the halogen reactor. The halogen reactor is operated at high temperature and pressure as the halogen digestion reaction is strongly exothermic. Heat removed from the halogen reactor (14) is typically recovered by heat exchangers (15) and distributed for use in the halogen electrolysis system or distributed to outside systems (10). Other products of the halogen digestion reaction include: (a) an ash (16) containing mineral compounds and materials not fully digested under the preferred operating conditions of the halogen reactor and (b) a mixture of hydrogen halide and water as in stream (41). The hydrogen halide and water are fed to an electrolyzer (12), which uses direct current electrical energy (9) to convert most of the hydrogen halide to hydrogen gas (8) and the dihalide. In the case of FIG. 2B, the hydrogen halide is HBr and the dihalide is Br₂. The dihalide is returned to the halogen reactor via a liquid stream (13), and excess water is exhausted from the electrolyzer.

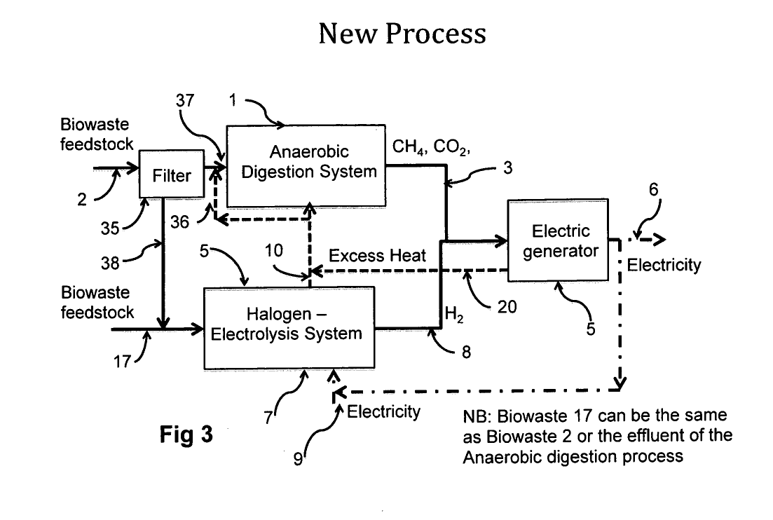

FIG. 3 shows the new process concept of this invention in overall block diagram form. Here, the known combination of anaerobic digestion and electrical generation is further combined with the halogen electrolysis system to substantially increase the amount of energy that may be recovered from a biowaste feedstock. First, a biowaste feedstock (2) is fed to a filter (35), where organic materials that are difficult for bacteria to digest are separated (38) and fed to the halogen digestion system (7). The separated biowaste (38) may be combined with the same or other biowaste feedstock (17) to provide more material for the halogen electrolysis process. The filtrate (37) containing biowaste that the bacteria in the anaerobic digester can handle is delivered to the anaerobic digester (1) for digestion. The halogen electrolysis system (5) converts its biowaste feedstock into excess heat for use in preheating (36) the feedstock (37) to the anaerobic digester, as well as heating the anaerobic digester itself. Hydrogen gas (8) from the halogen electrolysis system is combined with the biogas (3) from the anaerobic digester and is fed to the electric generator (5) for conversion into electricity and heat. The heat is returned (20) to the anaerobic digestion system and/or the halogen electrolysis system, and the electric energy is used in the halogen electrolysis system (9) and/or delivered (6) to uses outside the system of FIG. 3. This combined system concept is new in the art.

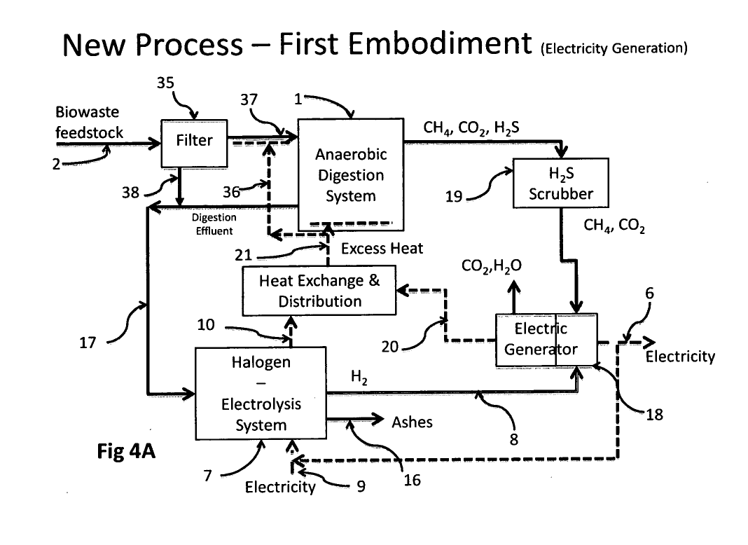

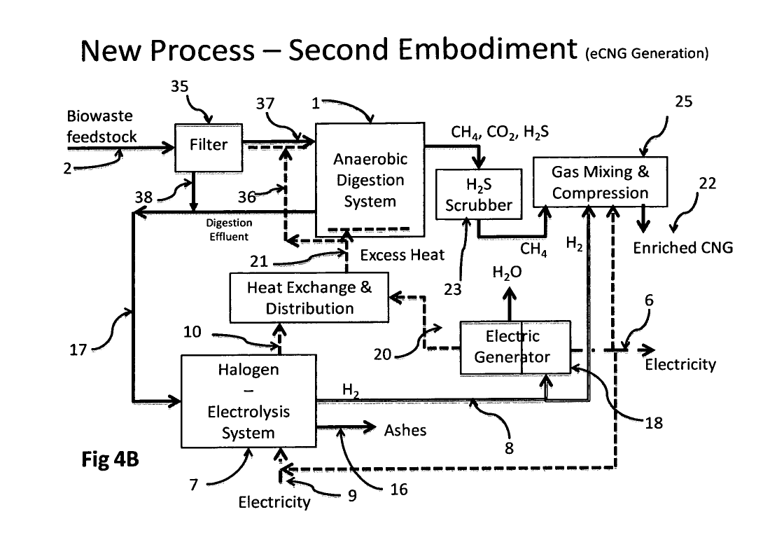

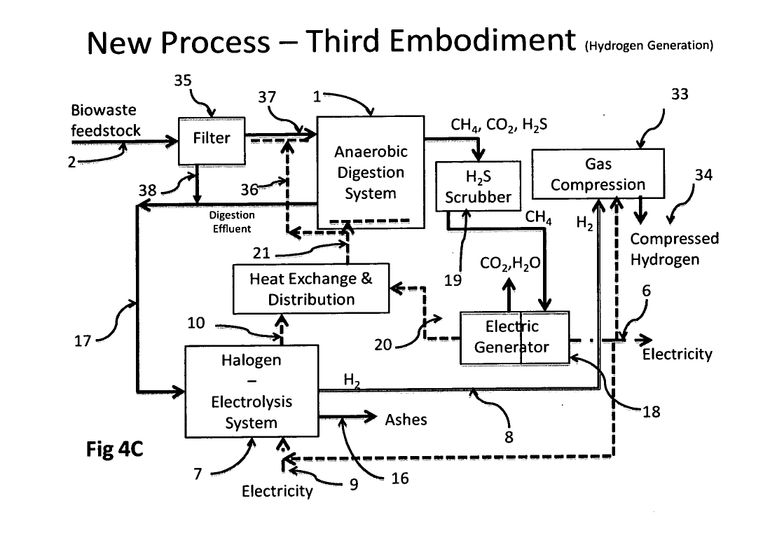

The biological digester (1) which is anaerobic in this example generates a substantial quantity of methane and carbon dioxide gas as well as sometimes a small amount of hydrogen sulfide. In a first embodiment shown FIG. 4A, the output gas stream (3) produced by the anaerobic digestion system is passed through a hydrogen sulfide scrubber (19) (or any equivalent process that will remove hydrogen sulfide from the gas stream). The resulting methane can, if desired, be mixed with the hydrogen produced by the halogen—electrolysis system and fed to an electricity generation system (18). The electric generator can be any number of electrical generation systems such as an internal combustion engine coupled with an alternator, but in a preferred implementation of the system, the electricity generation system is a solid oxide fuel cell that can use both methane and hydrogen to produce electricity with high efficiency. The heat generated from the halogen digester and the generator is sufficient to heat the anaerobic digestion feedstock and/or the digester sufficiently to enable anaerobic digestion even in cold climates as well as produce additional heat energy that can be used for other purposes.

In a second embodiment as shown in FIG. 4B, the methane is separated from the rest of the produced gases by a scrubber (23) or other equivalent process that can remove carbon dioxide and hydrogen sulfide from the gas stream (3). The methane produced is then mixed with the hydrogen gas (8) from the electrolysis unit (12) and compressed (25). The resultant compressed mixture of methane with the hydrogen gas (22) is an enriched compressed natural gas (CNG) that has 95% less emissions per Gallon Gasoline Equivalent (GGE) than diesel fuel. Many government transit fleets currently use compressed natural gas and commercial fleets are also evaluating use of this fuel. The combined effect of reducing the dependency on fossil fuels, whether foreign or domestic, reducing landfill for current biological wastes, abating methane generation to the atmosphere, and reducing the emissions from fleet vehicles is both economically and environmentally highly advantageous. Optionally, the gas mixture can be sent to the natural gas pipeline of the local utility.

In a third embodiment as shown in FIG. 4C the focus is on maximizing the production of hydrogen gas. The difference in this embodiment of our process is that the methane produced from the anaerobic digester (1) is sent through a scrubber (19) to remove any hydrogen sulfide present and then sent to the electrical generation system (20). This generated electricity is used to provide the power needs of the electrolysis system (12), the consumer's facility and can even be sold back to the electric utility grid. The gas output from the electrolysis system is compressed hydrogen gas (34).

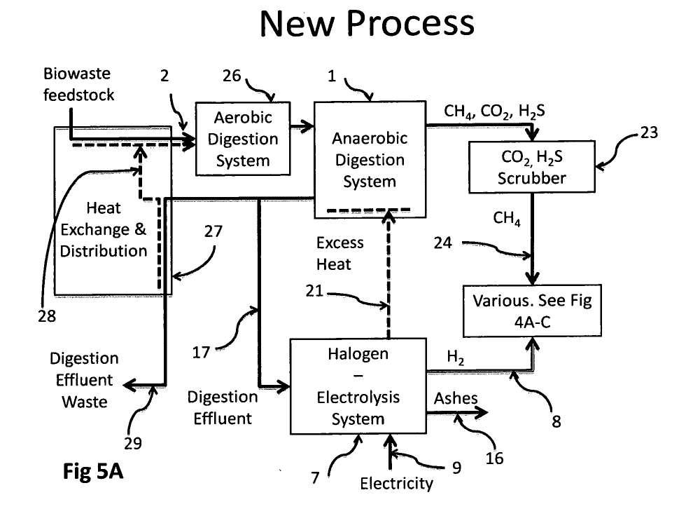

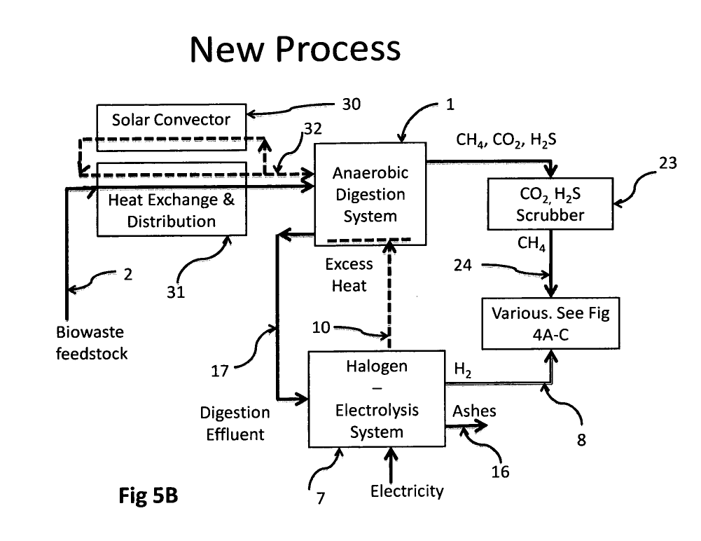

FIG. 5A describes another possible extension of our process targeted at optimizing its overall efficiency. This extension can be used in conjunction with any of the other embodiments described in FIGS. 3, 4A, 4B, and 4C. The main difference brought by this embodiment is that the biowaste feedstock (2) is fed first into an aerobic digester (26) before entering the anaerobic digester (1). The feedstock (2) enters the aerobic digester (26) at ambient temperature. The exothermic nature of the aerobic digestion increases the feedstock temperature and consequently brings the feedstock entering the anaerobic digester (1) to a temperature that is higher than the ambient temperature and consequently closer to the operating temperature required by the anaerobic digester (1). The higher temperature of the feedstock entering the anaerobic digester results in a reduction of the additional heat (21) needed by the anaerobic digester (1) to operate at optimal temperature. A lesser heat demand allows downsizing the heat exchange system and the halogen digestion system (7). The aerobic digestion process that occurs in the digester (26) also generates useful gazes such as methane that can increase the overall efficiency of the embodiment described in FIG. 5A.

For very cold climate where the ambient temperature is too low for the aerobic digester to operate efficiently, the digestion effluent (29) can be used to preheat the biowaste feedstock (2) through a heat exchanger (27). The heat exchanger (27) transfers the heat (28) recovered from the digestion effluent (29) that is exiting the anaerobic digester (1) at a temperature close to the operating temperature of the anaerobic digester (1) −32-40° C. for a mesophilic digestion for example—to the feedstock entering the heat exchanger at ambient temperature.

Other option covered by this specific embodiment but not showed on FIG. 5A includes the option to use part of the excess heat produced by any other components of the system (halogen digestion system, electricity generator) to preheat the feedstock (2).

FIG. 5B describes another variant of the embodiment presented in FIG. 5A where a solar convector (30) is used to collect heat from the sun. The solar convertor (30) is used in conjunction with a heat exchanger (31) to recover the heat generated by the sun (32) and use it to preheat the feedstock (2) before it enters the anaerobic digester (1). Optionally, the recovered heat (32) can also be used to provide additional heat to the anaerobic digester (1).

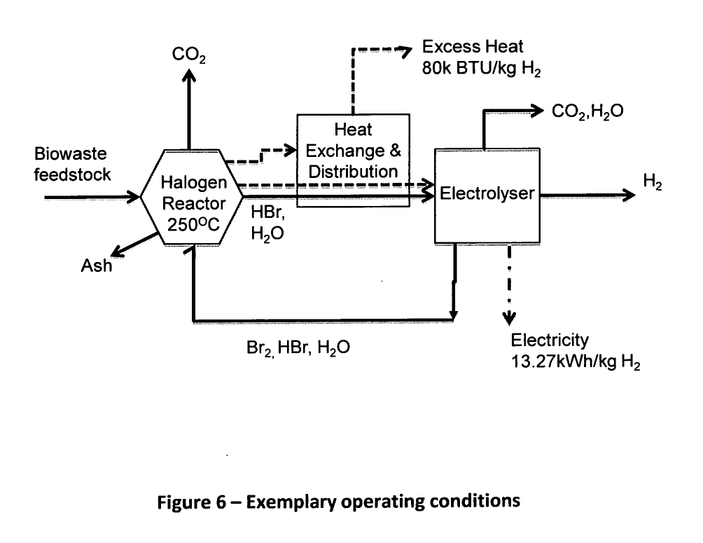

FIG. 6 shows the Halogen—Electrolysis System as used in the FIGS. 4 and 5 number (7). The two main components are the Halogen Reactor and the Electrolyzer. Feedstock is fed into the Halogen Reactor. It uses bromine to oxidize the feedstock. The output of this process is CO₂, sterile micronutrient ash, heat, H₂O, and hydrogen bromide. The heat is being distributed by the Heat Exchange & Distribution System. The hydrogen bromide is being treated in the Electrolizer chamber powered by electricity. The outputs of this process are the following streams: H₂, CO₂, H₂O, HBr, Br₂. The Br₂, HBr and H₂O are recycled into the Halogen Reactor. The H₂ is the valuable output of this process, the complete system is a hydrogen factory.

### Example 1

### Dairy Cow Operation

This example (described in the FIGS. 4A to 4C) outlines the process of the present invention as it is applied by way of example to a dairy operation comprising 500 cows. A 1400-pound dairy cow produces up to about 115 pounds of manure per day. This amount of manure, if left unmanaged, will release considerable amounts of methane (a much more potent greenhouse gas than CO₂). The waste from 500 cows introduces an estimated equivalent of 2750 tons of CO₂ per annum. Rather than contribute to greenhouse gases this manure can be advantageously used to provide usable energy for transportation, heat, and/or electricity.

An example of a conventional anaerobic digester is operating in a climate with an average temperature of 20 degrees Celsius. Due to the lower temperature the digester retention time is 45 days so the digester must be able to retain at least 3000 cubic meters of manure feedstock. The combination of the low average temperature and a widely variable temperature from day to night limits the yield from this digester to around 6000 cubic feet of methane per day. Even though the retention time is 45 days, there are still remains considerable solids in the effluent that will release methane into the atmosphere after it leaves the anaerobic digestion system.

This example can be best described by the embodiments in FIGS. 4A, 4B and 4C. Biodigesters for manure and known and are operating mainly in mild climates. In FIG. 4a the Anaerobic Digestion System (1) will receive heat (21) from the Halogen Electrolysis System (7) via a heat exchange system. This part of the present invention, allows higher and more stable temperatures to be maintained, thereby allowing for a much shorter retention time of 5 days. This equals a feedstock holding capacity requirement of around 340 cubic meters or a reduction of almost 90% compared to the lower, variable temperature of the current digester art. In addition, the methane yield is also increased to 15,000 cubic feet per day. The effluent (FIGS. 4A, 17) from the digester still contains approximately 40% of the biological solids of the original feedstock. Normally, this thermal potential is not utilized and the effluent (17) is either spread on fields or left to lie fallow. The energy remaining in the effluent (17) can be efficiently utilized by the Halogen Electrolysis System (7) of our invention. The halogen digester (part of the Halogen Electrolysis System (7)) uses bromine in this example to oxidize the remaining solids in the waste. The result is that heat (10) is created along with aqueous hydrogen bromide and a sterile micronutrient ash (16). The hydrogen bromide is converted by an electrolysis chamber (part of the Halogen Electrolysis System (7)) into hydrogen gas (8) and elemental bromine (which will remain in the Halogen Electrolysis System (7)). Depending on the heat needs of the digester (1) or the hydrogen needs, the amount of anaerobic digester effluent (17) processed can be readily adjusted. More effluent (17) can be diverted to the halogen digester when more heat and/or hydrogen are desired.

The hydrogen produced (8) can be utilized in a number of ways. If a transportation fuel is desired, then enough effluent can be all or partially consumed in the halogen digester to produce the hydrogen for mixing with methane to produce an enriched compressed natural gas that has 95% less emissions than diesel fuel. This process is described in FIG. 4b, where the Gas Mixing and Compression can be seen (25). If the hydrogen is consumed in a fuel cell or other highly efficient electricity generator as described in FIGS. 4A, 4B 4C (18), a net amount of electricity (6) can be generated over the electrolysis electricity needs (9). The large amount of heat generated can be also be used to enable the thermophilic digestion in climates of even the most extreme cold.

A known problem in the anaerobic digestion of manure today is the fibrous materials present in the feedstock (2). This does not digest well and increases retention times. Costs to handle and maintain digesters increases since the materials require more energy to stir and also build up which results in costly maintenance and down time for the digester. By filtering out (35) any fibrous material and sending it directly (38) to the chemical digester (7) the material is quickly dissolved and the resultant heat (10) and electricity can be used to drive the facility.

If the preferred output is to produce electricity then both the hydrogen and methane streams can be used in a fuel cell or other method of electricity generation. This system can produce an estimated net 180 KW continuous for the 500-cow dairy.

Regardless of the particular output selected, the final quantity of solids resulting from the process is greatly diminished with the majority of usable energy present in the manure feedstock efficiently harnessed. The output solids are also sterilized as a result of the oxidation process.

### Example 2

### Food and Beverage Waste. Spent Brewery Grain

This example as outlines the process of the present invention as it can be applied to a brewery operation that produces beer having an average alcohol content of approximately 5% and a capacity of 500 barrels of beer per day. At 80% brewery mash efficiency this equates to about 50 pounds of Brewers' Spent Grain (BSG) per barrel of beer, or around 11 metric tonnes of spent grain per day.

This process is best illustrated with the embodiment of FIG. 4B. Since the feedstock (2) (spent grain) needs no additional filtering, it can be fed into the Anaerobic Digester (1) after applying the heat (36) from the Halogen Electrolysis System (7) to facilitate the initial warming for thermophilic digestion. In this example the Halogen Electrolysis System (7) is fed by (17), which in this case is the effluent from the Anaerobic Digestion System (1) Once the available heat has been transferred, additional heat can be applied to the feedstock (2) (spent grain) if desired from the halogen digestion process. The final temperature of the BSG (2) would preferably be approximately 55° C., which is generally the optimal temperature for thermophilic anaerobic digestion to produce methane.

Once the grain is introduced into the anaerobic digester (1), it will normally take approximately 5 days before there are diminishing returns resulting from further retention of the feedstock. At this point, an estimated 45-60% of the organic solids have been consumed. The remaining energy in this effluent (17) can be harnessed by the halogen digestion process. The gas output would be an estimated 34,000 cubic feet of methane (CH4) produced per day.

If the focus of the system is for renewable transportation fuel (22) then the effluent (17) from the anaerobic digester (1) can be consumed in the Halogen Electrolysis System (7) to produce 24 kg of hydrogen (H₂) for mixing in the Gas Mixing and Compression System (25) with methane to produce the 330 gallons gasoline equivalent (GGE) of enriched compressed natural gas (22). This enriched compressed natural gas (22) has been proven to reduce emissions by 95% as compared to diesel fuel. The hydrogen/methane mixture also carries a 20% premium in value over conventional compressed natural gas itself.

Alternatively, the excess hydrogen (H₂) can be consumed in a fuel cell or other Electricity Generator (18) to offset the cost of the electricity required for the electrolysis of HBr to Br₂. If both the hydrogen and methane streams are used in an Electricity Generator (18) this can produce an estimated 700 KW continuously. This can generate enough electricity to power the entire system and plant operations with a large surplus that can be sold back to the electric utility company grid. The heat generated by both the halogen digester and the electricity generator can be used to facilitate the thermophilic anaerobic digestion, and/or brewery processes such as mashing of grains, boiling of wort, and/or used to cool the fermentation tanks for lagers. The energy needs of the entire brewery needs can be completely offset by this invention and have the benefit of income from the excess electricity sold and the reduction in disposal costs of the waste.

Using cellulose (C₆H₁₀O₅) as an example for spent grain, the following equations illustrate the Bromine digestion and regeneration process:

C₆H₁₀O₅+7H₂O+12Br₂→24HBr+6CO₂↑

2HBr→H₂↑+Br₂

We Claim:

1) A process for the conversion of biological feedstock into one or more combustible gases and nutrient rich solid comprising the steps of:
i) subjecting said feedstock to digestion in a biodigester by anaerobic bacteria to afford at least methane and an at least partially digested feedstock;
ii) reacting the at least partially digested feedstock with a halogen selected from the group consisting chlorine, bromine, iodine and mixtures thereof to produce heat, aqueous hydrogen halide and a micronutrient rich ash;
iii) electrolytically converting the hydrogen halide to gaseous hydrogen and molecular halogen.

2) A process for the conversion of biological feedstock into a nutrient rich solid comprising the steps of:
i) subjecting said feedstock to partial digestion by aerobic bacteria to afford an at least partially digested feedstock;
ii) subjecting said aerobically digested feedstock to further partial digestion by anaerobic bacteria;
iii) reacting the at least partially digested feedstock with a halogen selected from the group consisting chlorine, bromine, iodine and mixtures thereof to produce heat, aqueous hydrogen halide and a micronutrient rich ash;
iv) electrolytically converting the hydrogen halide to gaseous hydrogen and molecular halogen.

3) A process in accordance with claim 1 wherein the heat generated by step ii) is utilized to heat said feedstock prior to subjecting it to said digestion.

4) A process in accordance with claim 2 wherein the heat generated by step iii) is utilized to heat the feedstock.

5) A process in accordance with claim 1 wherein the heat generated by step ii) is utilized to heat other systems at the biological feedstock source facility.

6) A process in accordance with claim 1 wherein said digestion is carried out by at least one type of anaerobic bacteria.

7) A process in accordance with claim 6 wherein said anaerobic bacteria is a methanogenic bacteria.

8) A process in accordance with claim 6 wherein said anaerobic bacteria is a member of the Thermoanaerobacterium genus.

9) A process in accordance with claim 1 wherein the methane produced in step i) is combined with the hydrogen produced in step iii) and said admixture compressed for use as a fuel.

10) A process in accordance with claim 1 wherein the methane produced in step i) is used as a fuel.

11) A process in accordance with claim 9 wherein said admixture is combusted to power an electricity generator.

12) A process in accordance with claim 1 wherein the methane produced in step i) is combined with the hydrogen produced in step iii) and said admixture is converted into electricity and heat using a fuel cell.

13) A process in accordance with claim 10 wherein the methane produced is converted into electricity and heat using a fuel cell.

14) A process in accordance with claim 11 wherein the electricity generated is utilized to power the other processes at the biological feedstock source facility.

15) A process in accordance with claim 12 wherein the heat generated is utilized to heat the feedstock.

16) A process in accordance with claim 13 wherein the heat generated is utilized to heat the digester.

17) A process in accordance with claim 1 wherein the feedstock is heated to a temperature of at least about 55° C. prior to being brought into contact wherein said bacteria is thermophilic.

18) A process in accordance with claim 1 wherein the feedstock is heated to a temperature of at least about 32° C. prior to being brought into contact wherein said bacteria is mesophilic.

19) A process in accordance with claim 1 wherein the feedstock is heated to a temperature of at least about 12° C. prior to being brought into contact wherein said bacteria is psychrophilic.

20) A process in accordance with claim 1 wherein the hydrogen produced in step iii is separated for further processing.

21) An apparatus for the processing of biological feedstock comprising in operable combination:
i) an aerobic or anaerobic bacteria digestion unit;
ii) a halogen oxidation unit;
iii) an electrolysis unit for converting aqueous hydrogen halide produced in said halogen oxidation unit into molecular halogen and hydrogen gas.

22) An apparatus in accordance with claim 21 further comprising an electricity generating unit.

23) An apparatus in accordance with claim 22 where the electricity generating unit is a fuel cell.

24) A process in accordance with claim 1 wherein the feedstock is passed through a filter and divided into streams for diversion to the best suited digester, biological and/or chemical

25) An apparatus in accordance with claim 21 further comprising a heat exchanger.

26) An apparatus in accordance with claim 21 further comprising a compressor for compressing hydrogen gas, methane, or a combination thereof

27) An apparatus in accordance with claim 21 further comprising a scrubbing unit for collecting and separating out any carbon dioxide and/or hydrogen sulfide produced in said digestion unit.

28) An apparatus in accordance with claim 21 further comprising a mixing unit for combining methane produced in said digestion unit with hydrogen produced in said halogen oxidation unit.
